(19) United States
(12) Patent Application Publication
(10) Pub. No.: US 2007/0256713 A1
(43) Pub. Date: Nov. 8, 2007

(54) Method for Decontaminating Surfaces

(76) Inventors: Andrew J. Feitz, Coogee (AU); Trevor D. Waite, Wombarra (AU); Sung H. Joo, New Haven, CT (US)

Correspondence Address: OLSON & HIERL, LTD., 20 NORTH WACKER DRIVE, 36TH FLOOR, CHICAGO, IL, 60606, US

(21) Appl. No.: 10/581,391

(22) PCT Filed: Dec. 3, 2004
(86) PCT No.: PCT/AU04/01712

§ 371 Date: Feb. 21, 2007

(30) Foreign Application Priority Data
Dec. 4, 2003 (AU) 2003906724

(51) Int. Cl. A62D 3/00; A61L 9/014

(52) U.S. Cl. 134/42; 428/546

(57) ABSTRACT

A method for treating a surface to decontaminate the surface by chemically altering contaminants on the surface. The method comprises applying to the surface particles of a zero valent metal capable of reacting with oxygen and water to form hydroxyl radicals, and exposing the particles to oxygen and water. The method may be carried out in the presence of air.

### FIELD OF THE INVENTION

The present invention relates to a method for chemically altering contaminants on the surface of a solid substrate. The method may be used to decontaminate contaminated surfaces.

### BACKGROUND ART

The surface of a solid substrate can become contaminated with various contaminants. The contaminants may be non-toxic substances such as dirt, or substances harmful to humans, animals or the environment, such as toxic industrial pollutants (e.g. halogenated organic compounds), pesticides, herbicides, or chemical or biological warfare agents.

The contamination of the surface may limit the uses that may be made of the substrate. A contaminated surface may be decontaminated by treating the surface to remove the contaminants from the surface, or by treating the surface to destroy the contaminants or to convert the contaminants to other substances that have lesser impact on the intended use of the substrate.

When the contaminant is a relatively harmless substance, the contaminant may be removed from the surface by washing the surface with a liquid such as water or an organic solvent to wash the contaminant from the surface. However, washing a surface simply removes the contaminant from the surface with the liquid. For harmful contaminants, the liquid containing the contaminant washed from the surface may be difficult to dispose of safely or may itself need to be treated to remove the contaminant from the liquid. Further, washing the surface may riot remove a sufficient amount of a harmful contaminant from the surface to render the surface harmless to humans, animals or the environment.

The main methods used to decontaminate surfaces contaminated with harmful substances such as toxic industrial pollutants, pesticides or chemical or biological warfare agents, are neutralisation which involves chemically altering the contaminant to form less harmful products, and absorption which physically removes the contaminant from the surface but does not result in its destruction (e.g. using Fullers earth or activated charcoal).

Neutralisation is a preferred method for decontaminating surfaces contaminated with toxic substances as the method results in the destruction of the contaminant. For example, the most widely used method for neutralisation of chemical or biological warfare agents on a surface involves the use of hypochlorite to oxidise the contaminant into less harmful or harmless products. However, the use of hypochlorite to decontaminate surfaces has a number of disadvantages, including a reduction in active chlorine content in hypochlorite formulations over time and the corrosivity, flammability and toxicity of hypochlorite formulations.

Zero valent iron (ZVI) has been used for degrading organic contaminants (including chlorinated and nitro-substituted organic compounds) in groundwater. This degradation process involves the oxidation of Fe⁰ and the reduction of the organic contaminant. This degradation process is carried out under fully anoxic conditions since the presence of oxygen would prevent, or lower the efficiency of, the reduction of the contaminant.

A recent article has reported the oxidative destruction of chlorophenols in water in the presence of granular iron and EDTA, a strong iron binding ligand (Noradoun, C., et al (2003) “Destruction of Chlorinated Phenols by Dioxygen Activation under Aqueous Room Temperature and Pressure Conditions”, Ind. Eng. Chem. Res., 42, 5024-5030). That article reported that no oxidative degradation of the clorophenol in the presence of granular iron was observed (even after 70 hrs) in the absence of EDTA.

There remains a need for alternative methods for treating contaminated surfaces to remove the contaminant or chemically alter the contaminant.

It is to be understood that a reference herein to a prior art document does not constitute an admission that the document forms part of the common general knowledge in the art in Australia or in any other country.

### SUMMARY OF THE INVENTION

The present inventors have surprisingly found that particles of zero valent iron, or other zero valent metals capable of reacting with oxygen and water to form hydroxyl radicals, can be applied, in the presence of air, to surfaces contaminated with various contaminants, to chemically modify the contaminants on the surface.

In a first aspect, the present invention provides a method for treating a surface of a solid substrate to chemically alter contaminants on the surface capable of being oxidised by hydroxyl radicals, said method comprising applying to the surface particles of a zero valent metal capable of reacting with oxygen and water to form hydroxyl radicals, and exposing the particles to oxygen and water to form hydroxyl radicals at or near the surface.

In the method of the present invention, the contaminants on the surface are primarily chemically altered by oxidation of the contaminant, rather than reduction of the contaminant as is the case in the prior art anoxic processes for degrading contaminants in groundwater using zero valent iron.

The zero valent metal particles may be applied to the surface as dry particles. For example, the zero valent metal particles may be applied as a dry power. The zero valent metal particles can also be applied in admixture with a liquid carrier, such as an organic solvent, that evaporates during or shortly after application of the particles to the surface, leaving dry particles on the surface. When the zero valent metal particles are present on the surface as dry particles, the zero valent metal particles may be exposed to oxygen and water by exposing the particles to an atmosphere containing oxygen and water, such as air.

In some embodiments, the zero valent metal particles are exposed to water by applying the particles to the surface in admixture with water, or by applying water to the surface before, after or simultaneously with the application of the particles to the surface, so as to form a mixture of the particles and water on the surface. When the zero valent metal particles are applied to the surface in admixture with water, or water is applied to the surface before, after or simultaneously with the application of the particles to the surface, to form a mixture of the particles and water on the surface, the particles are typically exposed to oxygen by exposing the mixture of the particles and water to an atmosphere containing oxygen, typically air.

Typically, the particles are applied to the surface and simultaneously exposed to oxygen and water, by applying the particles to the surface in the presence of an atmosphere containing oxygen and water vapour, typically air.

Typically, the zero valent metal capable of reacting with oxygen and water to form hydroxyl radicals is zero valent iron or zero valent copper, more typically zero valent iron. However, other zero valent metals capable of reacting with oxygen and water to form hydroxyl radicals could be used.

Typically, the particles of the zero valent metal have an average primary particle size in the range of from 0.5 to 3000 nm.

In some embodiments, the zero valent metal particles are applied to the surface as a powder. In other embodiments, the zero valent metal particles are applied to the surface in admixture with a liquid carrier, typically water.

In a second aspect, the present invention provides a method for treating a surface of a solid substrate to chemically alter contaminants on the surface capable of being oxidised by hydroxyl radicals, the method comprising applying to the surface a mixture comprising water and particles of zero valent iron or zero valent copper in the presence of air, wherein the particles of zero valent iron or zero valent copper have an average primary particle size in the range of from 0.5 to 3000 nm.

In a third aspect, the present invention provides particles of a zero valent metal when used in the method of the first or second aspects of the present invention.

### BRIEF DESCRIPTION OF THE DRAWINGS

FIG. 1 is a graph of the relative concentration (C/C₀) of molinate versus time in a 50 mL suspension containing, at time zero, 100 ppb molinate and 10.7 mM of nano-sized zero valent metal particles (nZVI) and having a pH of 4, and sparged with N₂ (♦), zero-grade air (●) or 100% O₂ (∘) or shaken in the presence of air (□). Also shown is a control in which a ZVI-free solution containing 100 ppb molinate is sparged with N₂ (Δ).

FIG. 2 is a graph of the dissolved oxygen concentration (DO) versus time in a 50 mL suspension containing, at time zero, 100 ppb molinate and 1.8 mM nZVI, 5.4 mM nZVI or 10.7 mM nZVI shaken in the presence of air.

FIG. 3 is a graph of the relative concentration (C/C₀) of molinate versus time in a 50 mL suspension containing, at time zero, 100 ppb molinate and 0.9 mM nZVI and having a pH of 8.0 shaken in the presence of air.

FIG. 4 is a graph of the para-hydroxybenzoic acid (p-HBA) concentration versus time for two 50 mL suspensions containing, at time zero, 0.9 mM nZVI and 10 mM benzoic acid and having 30 mM ionic strength shaken in the presence of air. One suspension had a pH of 3 (Δ) and the other had a pH of 8 (•).

FIG. 5 shows a graph of the p-HBA concentration and yield of oxidants ([p-HBA]×5.87 per mole of nZVI) versus nZVI concentration after 1 hour in a 50 mL suspension shaken in the presence of air, the suspension having an initial concentration of 10 mM benzoic acid and 0.2, 0.9, 1.8, 3.6 or 5.0 mM nZVI particles, a pH of 3 and 30 mM ionic strength.

### DETAILED DESCRIPTION OF THE INVENTION

The method of the present invention comprises applying particles of a zero valent metal to the surface, and exposing the particles to oxygen and water to form hydroxyl radicals at or near the surface.

The particles of the zero valent metal may be exposed to oxygen and water by any means that brings the particles into contact with oxygen and water.

The particles of the zero valent metal may be exposed to oxygen and water by exposing the particles to an atmosphere containing oxygen and water, such as air. When the particles are exposed to an atmosphere containing oxygen and water, the particles contact oxygen and water from the atmosphere.

Typically the particles are applied to the surface in the presence of an atmosphere containing oxygen and water vapour. Typically the particles are applied to the surface in the presence of air. However, the particles could be applied to the surface in the presence of an artificial atmosphere containing oxygen and water vapour.

Preferably, the zero valent metal particles are applied to the surface in admixture with water, or water is applied to the surface before, after or simultaneously with the application of the particles to the surface, to form a thin film of a mixture of the particles and water on the surface. When the particles are applied to the surface in this manner, and the mixture of the particles and the water exposed to an atmosphere containing oxygen, such as air, the particles are contacted with oxygen in the atmosphere and/or oxygen dissolved in the water. Typically, the zero valent metal particles are applied to the surface in a mixture with water. The inventors have found that when the particles of the zero valent metal are in contact with liquid water, the rate of formation of hydroxyl radicals is greater the higher the concentration of oxygen in the water in contact with the particles. Thus when the particles are applied to the surface together with water so as to form a thin film of a mixture of the particles and water on the surface in the presence of air, the rate of formation of hydroxyl radicals in the thin film is greater than if the particles had been placed in a container of water exposed to air.

When the zero valent metal particles are applied to the surface and exposed to oxygen, for example from the surrounding atmosphere, the zero valent metal is rapidly oxidised by oxygen (in the case of zero valent iron, the Fe⁰ is oxidised to form Fe(II)). The oxygen accepts electrons from the metal and forms hydrogen peroxide either directly or via a reduced form of oxygen (superoxide). In the presence of water, for example from the surrounding atmosphere, the superoxide self terminates to form hydrogen peroxide. The hydrogen peroxide reacts with the metal ion produced by the oxidation of the zero valent metal, to form highly oxidative hydroxyl radicals. These radicals are highly reactive and react with contaminants, oxidising the contaminant. When the contaminant is a toxic organic compound such as halogenated organic compounds, a chemical warfare agent or a biological warfare agent, the product of the oxidation of the contaminant by the hydroxyl radical is typically less harmful than the contaminant thus leading to the decontamination of the surface. Oxidation of the contaminant by the hydroxyl radical is the primary mechanism of chemically altering contaminants on the surface in the method of the present invention. However, some of the contaminants on the surface may be chemically altered by reaction with the zero valent metal itself resulting in a reduction of the contaminant, or by reaction with the hydrogen peroxide oxidising the contaminant.

Without wishing to be bound by theory, it is believed that for zero valent iron, the reactions resulting in the formation of the hydroxyl radicals are as set out below (where “>” represents adsorbed species):
Fe⁰+O₂→>O₂ (adsorption of oxygen onto iron particles)
Fe⁰+>O₂→>Fe²⁺+2>e⁻ (corrosion of iron)
>O₂+e⁻→>O₂^{•-} (reaction of adsorbed O₂ with electrons)
>Fe²⁺→Fe²⁺ (release of Fe²⁺ from iron particles into surrounding water)
O₂^{•-}+O₂^{•-}+2H⁺→H₂O₂+O₂ (formation of hydrogen peroxide—this reaction may occur between adsorbed or released superoxide)
Fe²⁺+H₂O₂→FeOH²⁺+OH• (Fenton reaction)

A similar Fenton-like reaction occurs between other metal ions such as Cu⁺and hydrogen peroxide to form hydroxyl radicals.

The particles of the zero valent metal may be any particles of the zero valent metal. Preferably the particles have an average primary particle size in the range of from 0.5 to 3000 nm. As used herein, a reference to “nano-sized” particles refers to particles having an average primary particle size in the range of from 0.5 to 3000 nm. Such particles are preferred as the larger surface area of the particles per unit weight compared to particles having a larger particle size, results in a more rapid oxidation of the metal and thus more rapid formation of hydroxyl radicals. More preferably, the particles have an average primary particle size in the range of from 5 to 2000 nm, more preferably from 10 to 1500 nm, more preferably from 10 to 500 nm and more preferably from 10 to 200 nm.

Nano-sized zero valent iron particles are typically in the form of an aggregate of the primary particles. Typically, the average aggregate (secondary) particle size of nano-sized zero valent iron particles is in the range of from 0.1 to 10 μm, more preferably, from 0.2 to 5 μm, more preferably from 0.3 to 3 μm, and more preferably from 0.5 to 2 μm.

Nano-sized zero valent metal particles may be prepared by methods known in the art. For example, nano-sized zero valent iron particles may be synthesised by reduction of an aqueous solution of Fe²⁺ or Fe³⁺ using a strong reducing agent such as sodium borohydride. This produces nano-sized zero valent iron particles having a primary particle size of 1-200 nm. Similarly, nano-sized zero valent copper particles may be synthesised by reduction of an aqueous solution of Cu²⁺ using a strong reducing agent such as sodium borohydrate.

The zero valent metal particles may be applied to the surface by any means. Nano-sized zero valent metal particles may for example be applied to the surface as a dry powder. However, preferably, the zero valent metal particles are applied to the surface by applying to the surface a mixture comprising the zero valent metal particles and water, e.g. a mixture comprising a colloidal suspension of the particles in water.

When the zero valent metal particles are nano-sized particles, the mixture comprising the particles and water is typically applied to the surface by spraying the mixture on to the surface. Because of the size of the nano-sized zero valent metal particles, a dispersion of the nano-sized zero valent metal particles in water can typically be sprayed on to the surface using conventional equipment used for spraying aqueous formulations on to a surface.

In some embodiments, the mixture comprising the zero valent metal particles and water is substantially free of components other than the zero valent metal particles and water. However, in other embodiments, the mixture may comprise one or more additional agents. In some embodiments, the mixture may comprise one more agents selected from the group consisting of stabilisers, co-solvents and surfactants. In some embodiments, the mixture may comprise one or more thickeners, foaniers or viscosity enhancers to promote adherence of the zero valent metal particles to a vertical surface and/or to encapsulate desorbing vapour. In some embodiments, the mixture is substantially free of strong metal binding ligands such as EDTA. Typically the pH of the mixture is from 2 to 8, for example between 2 and 4.

Prior to use in the method of the present invention, the zero valent metal particles can conveniently be stored as a mixture with water in the absence of oxygen or any other species that can oxidise the zero valent metal. Such a mixture may for example contain 10 g to 100 g of the zero valent metal particles per litre. In this form, the zero valent metal particles are non-toxic, non-flammable, non-corrosive and non-hazardous. When used to treat a contaminated surface, this mixture can be applied to the surface without modification using equipment, such as the Titan “Spraysafe” equipment, conventionally used to apply aqueous formulations to a surface.

The surface may be the surface of any solid substrate. The substrate may for example be a building, part of a building, furniture, machinery, vehicles such as motor vehicles, tanks or aircraft, or terrain such as a road, pathway or soil. The substrate may for example be personal equipment such clothing, protective clothing, protective glasses, helmets, hats etc. The substrate may for example be made of wood, glass, metal (e.g. steel, aluminium, etc), plastic, a fabric, or may be a painted surface. Preferably the substrate is formed of a material that is less reactive with hydroxyl radicals than the contaminant.

The contaminant may be any substance that has come to be in contact with the surface and that is capable of being oxidised by hydroxyl radicals. The contaminant may be an organic contaminant or an inorganic contaminant (e.g. arsenic (III) which can be oxidised to arsenic (V)). Typically, the contaminant is an organic contaminant. The contaminant may for example be a toxic industrial pollutant such as a halogenated organic compound (e.g. a chlorinated aliphatic, chlorinated aromatic and/or polychlorinated biphenyl compound), a pesticide or a herbicide. The contaminant may be a chemical or biological warfare agent. The chemical warfare agent may for example be one of the vesicant class of agents, such as sulphur mustard, nitrogen mustard and lewisite, or a nerve agent such as Tabun, Sarin, Soman or VX. The contaminant may be a toxin produced by biological organisms, such as the plant toxin ricin. The contaminant may also be a microorganism such as the potential biological warfare agents anthrax, Q-fever or Venezuelan equine encephalitis.

When toxic organic contaminants such as halogenated organic compounds, pesticides, herbicides, or chemical warfare agents are oxidised by the hydroxyl radicals, the oxidation product is typically harmless or less harmful to humans, animals and the environment than the organic contaminant. Depending on the intended use of the substrate and the toxicity of the oxidation products, the oxidation products may be allowed to remain on the surface, may be removed from the surface (e.g. by washing the surface or by adsorption with an agent such as Fullers earth or charcoal) or the surface may be further treated to chemically alter the oxidation products.

The method of the present invention can be used to treat surfaces contaminated with a variety of contaminants. The method of the present invention can be carried out in the presence of air, and therefore can be used to treat surfaces of large fixed structures such as buildings, or terrain such as roads. As the method can be carried out in the presence of air, the method can be used to treat surfaces without requiring a specialised environment for the treatment process, and therefore can be used to treat the surfaces of equipment, clothing etc in the field.

The method advantageously does not require light or an external energy source to initiate the reactions leading to the chemical alteration of the contaminant. The method also advantageously does not require the presence of a strong metal binding ligand. Accordingly, in various embodiments of the invention, the zero valent metal particles are applied to the surface and exposed to oxygen and water without the presence of a strong metal binding ligand, such as EDTA.

In preferred embodiments, the particles of a zero valent metal capable of reacting with oxygen and water to form hydroxyl radicals are nano-sized zero valent iron particles. When nano-sized zero valent iron particles are applied to the surface of a porous substrate and exposed to oxygen and water, the nano-sized zero valent iron particles and the produced hydroxyl radicals can penetrate into the surface of the porous substrate, and the hydroxyl radicals are therefore able to react with contaminants adsorbed onto the surface and absorbed into the surface. The large surface area of nano-sized zero valent iron particles allows for a rapid oxidation of the iron resulting in rapid formation of hydroxyl radicals and therefore oxidation of the contaminants. A further advantage of the method of the present invention using nano-sized zero valent iron particles is that the ultimate oxidation product of the zero valent iron particles is nano-sized ferrihydrate particles. These particles have a large surface area and are capable of adsorption and hydrolysis of by-products of the oxidation of the contaminants.

In one particularly preferred embodiment, the particles of a zero valent metal capable of reacting with oxygen and water to form hydroxyl radicals are nano-sized zero valent iron particles having an average primary particle size in the range of from 10 to 200 nm, and the nano scale zero valent iron particles are applied to the surface by spraying onto the surface a suspension of the zero valent iron particles in water, the suspension comprising from 10 to 100 g of the particles per litre, in the presence of air. The nano-sized zero valent iron particles for use in this embodiment may be prepared as described in the experimental section below.

### EXPERIMENTAL

Controlled experiments were conducted to show how oxygen availability affects the oxidation of organic contaminants using nano-sized zero valent iron particles (nZVI). In order to assess the effects of oxygen availability, the degradation of the herbicide molinate was examined under deoxygenated conditions, in the presence of air and in the presence of 100% oxygen. Further experiments were conducted using benzoic acid to examine the oxidisation of organic compounds in a suspension of nZVI exposed to the air. Benzoic acid was used as a probe compound in these experiments as the amount of the oxidisation products (isomers of hydroxybenzoic acid) can be readily measured by measuring the amount of p-hydroxybenzoic acid (p-HBA) produced.

### Materials and Methods

Reagents

Molinate (99% purity) was purchased from Alltech Associates (Aust) Pty Ltd. Molinate solutions were prepared from a 100 ppm stock solution. All other chemicals used in this work were analytical reagent grade. Solutions of molinate and benzoic acid were prepared in ultra-pure water (Milli-Q water, Millipore).

Manufacture and Characterization of Nanoscale Zero Valent Iron

Colloidal zero valent iron particles were synthesized by adding an aqueous solution of 0.16M NaBH₄ (98%, Aldrich) drop-wise to a 0.1M FeCl₃·6H₂O (98%, Aldrich) solution at ambient temperature with magnetic stirring. Ferric iron is reduced and Fe₀ precipitated according to the following reaction:
Fe(H₂O)₆³⁺+3BH₄⁻+3H₂O→Fe⁰(s)+3B(OH)₃ +10.5H₂

The freshly synthesized Fe⁰ particles so produced were washed with 10⁻⁴M HCl three times before storing in 10⁻⁴M HCl at a concentration of 5 mg Fe/mL. The Fe⁰ particles are present as a suspension in the 10⁻⁴ M HCl, and can be stored in this form in an anoxic environment, for example, in a sealed container, for extended periods prior to use. Single point adsorption BET analysis indicated that the nZVI particles produced in this way have a surface area of 32 m²/g. The size of individual particles ranged from 1-200 nm with an average primary particle size of 50 nm.

Analysis of Molinate

Solid-phase microextraction (SPME) followed by GC/MS (HP 19091S-433) was used to determine molinate concentrations. All samples were filtered using AP20 glass-fibre syringe filters before analysis. A polydimethylsiloxane (PDMS) 100 μm SPME fibre was conditioned before initial application in the injection port of the GC by heating it at 250° C. for 2 hours according to instructions provided by the supplier (Supelco). For the SPME process, 2 mL vials were filled with 1 mL of aqueous samples containing molinate and extracted onto the fibre for 15 min under rapid stirring conditions. The fibre was removed from the sample and introduced into the GC/MS injector where molinate was thermally desorbed for 3 min and injected onto a (HP-5MS) column in splitless mode with the injector held isothermally at 260° C. For quantification of molinate, the GC/MS was operated in the SIM (Selected Ion Monitoring) mode, by monitoring the base peak of molinate. The temperature program in SIM mode was as follows: the initial temperature was 80° C. (1 min), which was increased to 178° C. (3 min) at 30° C./min, and then to 250° C. (5 min) at 30° C./min, giving total run time of 14.7 min. The detector was set at 280° C., helium (pure carrier gas grade) was used as the carrier gas at a flow-rate of 1.0 mL/min and the electron impact (EI) ionization energy was set to 70 eV. All standard curves involved use of five concentrations and were linear with regression coefficients greater than 0.9995 in all cases. The method provided a limit of detection for molinate of 10 ng/L. In order to prevent carryover, blanks were run before the next sample extraction. To account for the filter recovery (95%), initial samples (before adding nZVI) were also filtered.

Analysis of P-hydroxybenzoic Acid (p-HBA)

p-HBA concentrations were determined by high-performance liquid chromatography (HPLC) using a Hewlett-Packard 1100 series HPLC system equipped with a 250×4.6 mm Waters Spherisorb ODS-25μcolumn (Alltech, IL). A two-solvent gradient elution, consisting of water (pH 3) and acetonitrile (85:15, v/v %) at a flow rate of 1.0 mL/min was used to separate beiizoic acid (BA) and isomers of hydroxybeiizoic acid. The p-HBA isomer was quantified at 255 nm and BA was quantified at 270 nm. All standard curves were linear with regression coefficients of >0.9990 in all cases. The method detection limit for BA and p-HBA were 2.5 μM and 0.1 μM respectively.

Batch Experimental Setup

Experiments were carried out at room temperature (20 ±2° C.) under an air atmosphere without pH control (pH˜6-7) as well as at initial pH values of 4 (obtained by HCl addition) and 8.0 (obtained using a 2 mM bicarbonate buffer) for the molinate experiments. Benzoic acid experiments were conducted at pH 3 or pH 8 (controlled through addition of HCl or bicarbonate buffer) with a benzoic acid concentration of 10 mM. Experiments were conducted in 100 mL serum boffles using a total suspension volume of 50 mL under aerobic conditions. The 50 ml suspensions were prepared by preparing a solution of molinate or benzoic acid in water, and adding nZVI from the 5 mg Fe/mL stock suspension prepared as described above. The bottle containing the suspension was continuously shalen open to air atmosphere at 175 rpm using an orbital shaker (Hybritech Incorporated), or sparged with zero grade air, pure oxygen or nitrogen for the duration of the experiment.

Results and Discussions

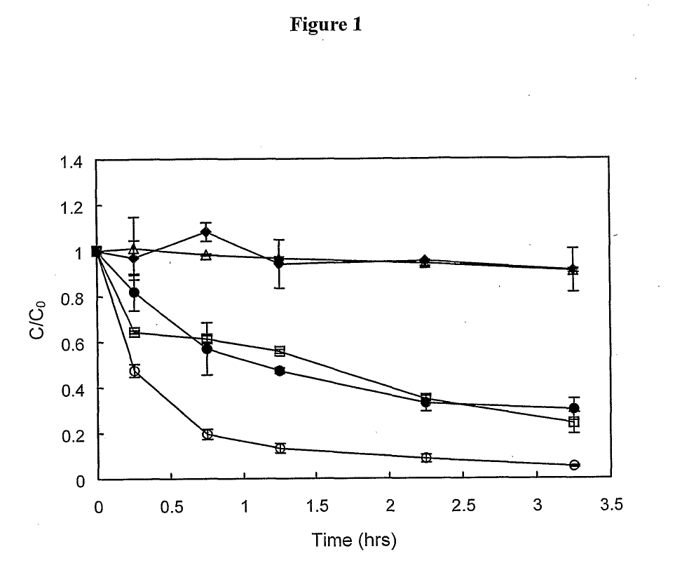

In a first experiment, a 50 mL suspension was prepared containing 100 ppb molinate and 10.7 mM nZVI and having an initial pH of 4, and the relative concentration of molinate over time measured while identical suspensions were sparged with N₂, zero-grade air or 100% O₂ or shaken in the presence of air. As a control, a ZVI-free solution containing 100 ppb molinate was sparged with N₂ and the relative concentration of molinate over time measured. The results are shown in FIG. 1.

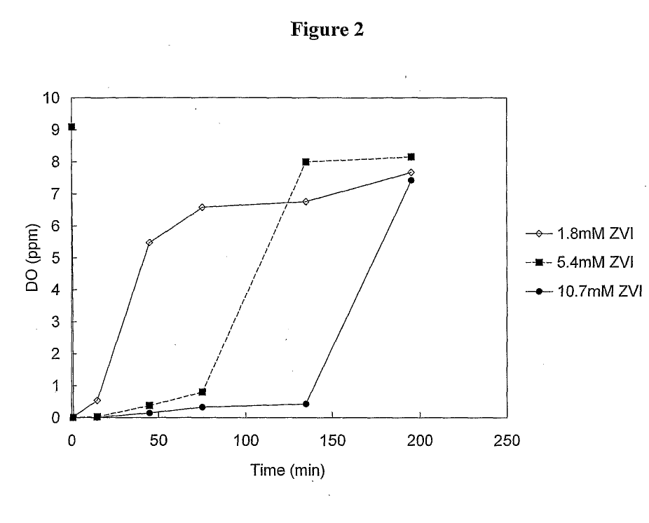

In a second experiment, 50 mL suspensions containing 100 ppb molinate and 1.8 mM nZVI, 5.4 mM nZVI or 10.7 mM nZVI were prepared, and the amount of dissolved oxygen in ppm measured over time while the suspension was shaken in the presence of air. The results are shown in FIG. 2.

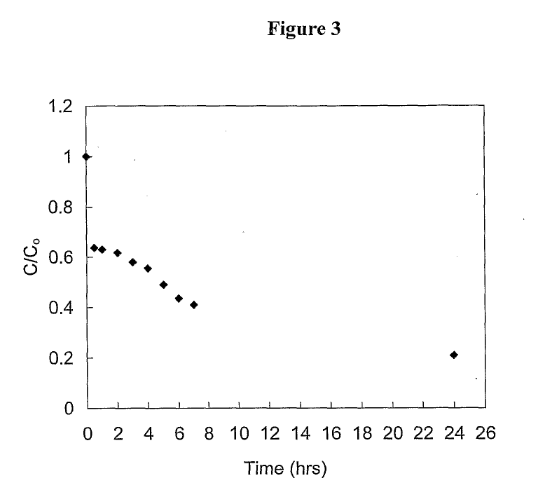

In a third experiment, a 50 mL suspension containing 100 ppb molinate and 0.9 mM nZVI and having a pH of 8.0 was prepared, and the concentration of molinate relative to the initial concentration was measured over time while the suspension was shaken in the presence of air. The results are shown in FIG. 3.

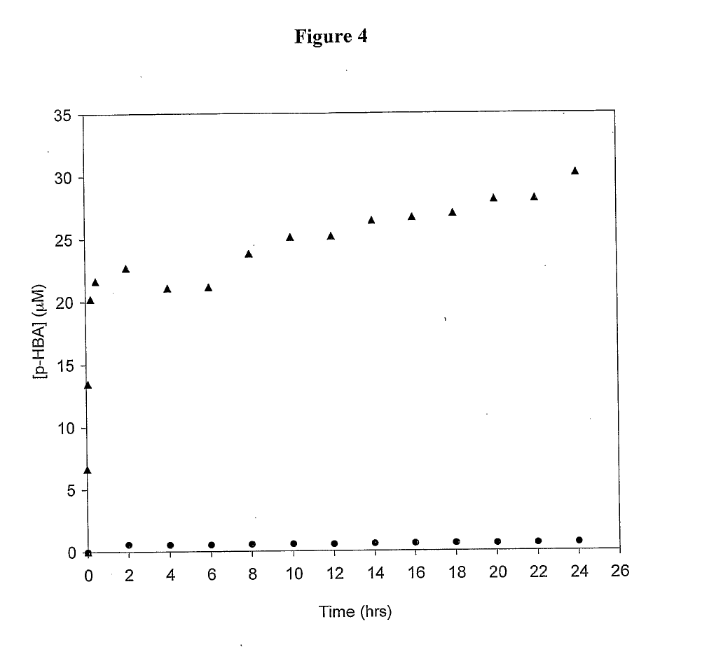

In a fourth experiment, a 50 mL suspension containing 0.9 mM nZVI and 10 mM benzoic acid and having 30 mM ionic strength and a pH of 3 was prepared, and the p-HBA concentration in the suspension over time measured while the suspension was shaken in the presence of air. A second suspension containing 0.9 mM nZVI and 10 mM benzoic acid and having 30 mM ionic strength and a pH of 8 was prepared, and the p-HBA concentration over time measured while the suspension was shaken in the presence of air. The results are shown in FIG. 4.

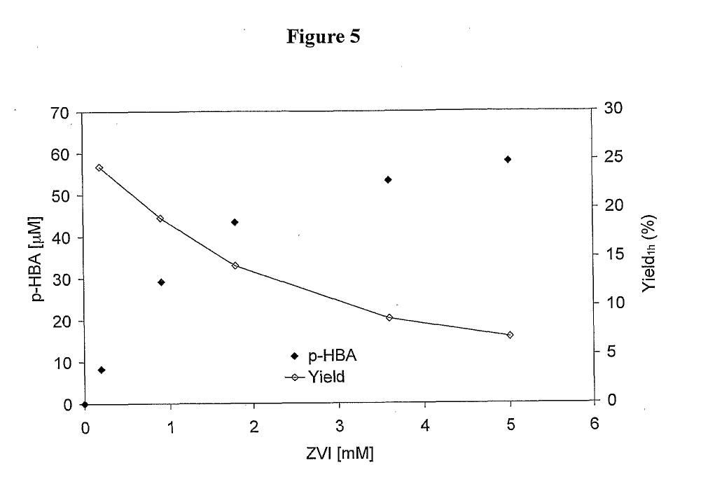

In a fifth experiment, 50 mL suspensions were prepared having an initial concentration of 10 mM benzoic acid and 0.2, 0.9, 1.8, 3.6 or 5 mM nZVI particles, a pH of 3 and 30 mM ionic strength. The suspensions were shaken in the presence of air and the concentration of p-HBA measured after 1 hour. The results are shown in FIG. 5.

In FIGS. 1 to 4, time zero is the time at which the suspension was formed, that is, the time when the nZVI was added to the molinate or benzoic acid solution.

As shown in FIG. 1, molinate showed little removal in the absence of oxygen while 70% removal was observed over 3 hours when the sample was gently sparged with air. Supersaturating the suspension by sparging with pure oxygen resulted in a faster degradation rate. Vigorous shaking of the serum bottles under air atmospheric conditions achieved a similar degree of removal as gently sparging with zero-grade air. No removal of molinate from the suspension is observed when sparging with nitrogen gas, indicating that molinate is not adsorbed to the nZVI particles and is not reductively degraded.

Addition of nZVI to the molinate solution instantly consurnes the dissolved oxygen in the solution (FIG. 2) and results in a release of ferrous iron, the production of hydrogen peroxide and an increase in the pH.

A striking feature of degradation using nZVI is the biphasic kinetics due to oxygen limitations. As can be seen from FIG. 3, fast removal of molinate is initially observed with slower, sustained degradation over the remaining 24 hours. The results of studies into the nZVI-mediated degradation of benzoic acid yield similar biphasic kinetics with respect to the generation of the oxidation product, p-hydroxybenzoic acid (FIG. 4). Benzoic acid was transformed into three isomers of hydroxybenzoic acid. Although it was not possible to quantify each of the isomers due to difficulty resolving the ortho and meta forms, the three isomers of hydroxybenzoic acid account for 90 ±5% of the products of OH¹⁰⁸ reactions with BA with the ratio of o-HBA, m-HBA, and p-HBA products reported to be in the proportion 1.7: 2.3: 1.2. After an initial pulse of radical production and accumulation of p-HBA during the first 15 minutes, the amount of p-HBA in the suspension slowly increases over 24 hours. Considerably more p-HBA is generated at pH 3 compared to pH 8. The reason for the lower yield at pH 8 can be attributed to faster passivation at higher pH and that Fe²⁺ is oxidised quickly by O₂ at this pH.

The initially fast removal of molinate and evolution of p-HBA from the benzoic acid system coincides with the rapid disappearance of dissolved oxygen. After initial consumption of dissolved oxygen, ongoing degradation is limited by oxygen diffusion into the suspension. In competition with oxygen diffusion is the formation of a passivation layer on the particle surface. Hence the fast initial molinate removal can be attributed to the pulse of hydroxyl radicals that are generated on exposure of the nZVI particles to the oxygen containing solutions.

If oxygen is not maintained in excess during this time, the rate of oxidation is limited by oxygen diffusion into the suspension and competition from oxide formation on the particle surface. Studies with benzoic acid reveal that the initial hydroxyl production efficiency can be as high as 25% of the added nZVI at pH 3 (FIG. 5). The highest initial reaction efficiency was observed at the lowest nZVI concentration when dissolved oxygen is in excess (i.e. 0.26 mM O₂ and 0.2 mM nZVI). Therefore maintaining an environment where oxygen is in excess or oxygen transfer into the water surrounding the nZVI is fast, i.e through a thin film of water and nZVI particles on a surface, will facilitate a greater initial pulse of oxidants and a higher oxidant yield.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method for treating a surface of a solid substrate to chemically alter contaminants on the surface capable of being oxidised by hydrokyl radicals, the method comprising applying to the surface particles of a zero valent metal capable of reacting with oxygen and water to form hydroxyl radicals, and exposing the particles to oxygen and water to form hydroxyl radicals at or near the surface.

2. The method according to claim 1, wherein the zero valent metal particles are applied to the surface as dry particles.

3. The method according to claim 2, wherein the zero valent metal particles are exposed to oxygen and water by exposing the zero valent metal particles to the air.

4. The method according to claim 1, wherein the zero valent metal particles are applied to the surface by applying to the surface a mixture comprising the zero valent metal particles and water.

5. The method according to claim 4, wherein the zero valent metal particles have an average primary particle size in the range of from 0.5 to 3000 nm, and wherein the mixture comprising the particles and water is sprayed onto the surface.

6. The method according to claim 1, wherein water is applied to the surface before, after or simultaneously with the application of the zero valent metal particles to the surface, so as to form a mixture comprising the zero valent metal particles and water on the surface.

7. The method according to any one of claims 4 to 6, wherein the zero valent metal particles are exposed to oxygen by exposing the mixture comprising the zero valent metal particles and water to the air.

8. The method according to any one claims 1 to 7, wherein the zero valent metal particles are applied to the surface in the presence of air.

9. The method according to any one of claims 1 to 8, wherein the zero valent metal capable of reacting with oxygen and water to form hydroxyl radicals is zero valent iron or zero valent copper.

10. The method according to any one of claims 1 to 9, wherein the zero valent metal capable of reacting with oxygen and water to form hydroxyl radicals is zero valent iron.

11. The method according to any one of claims 1 to 4 or 6 to 10, wherein the zero valent metal particles have an average primary particle size in the range of from 0.5 to 3000 nm.

12. The method according to any one of claims 1 to 10, wherein the zero valent metal particles have an average primary particle size in the range of from 5 to 2000 nm.

13. The method according to any one of claims 1 to 10, wherein the zero valent metal particles have an average primary particle size in the range of from 10 to 1500 nm.

14. The method according to any one of claims 1 to 10, wherein the zero valent metal particles have an average primary particle size in the range of from 10 to 500 nm.

15. A method for treating a surface of a solid substrate to chemically alter contaminants on the surface capable of being oxidised by hydroxyl radicals, the method comprising applying to the surface a mixture comprising water and particles of zero valent iron or zero valent copper in the presence of air, wherein the particles of the zero valent iron or zero valent copper have an average primary particle size in the range of from 0.5 to 3000 nm.

16. The method according to claim 15, wherein the particles of zero valent iron or zero valent copper have an average primary particle size in the range of from 5 to 2000 nm.

17. The method according to claim 15, wherein the particles of zero valent iron or zero valent copper have an average primary particle size in the range of from 10 to 1500 nm.

18. The method according to claim 15, wherein the particles of zero valent iron or zero valent copper have an average primary particle size in the range of from 10 to 500 nm.

19. Particles of a zero valent metal when used in the method of any one of claims 1 to 18.
